(19) United States
(12) Patent Application Publication
(10) Pub. No.: US 2024/0173198 A1
(43) Pub. Date: May 30, 2024

(54) MASSAGER CAPABLE OF ADJUSTING RANGE OF MOTION OF MASSAGING MEMBER

(71) Applicant: Dongguan Duoguo Zhichuang Technology Co., Ltd., Dongguan City (CN)

(72) Inventor: Changzhi Luo, Dongguan City (CN)

(21) Appl. No.: 18/430,268

(22) Filed: Feb. 1, 2024

(30) Foreign Application Priority Data
Nov. 15, 2023 (CN) 202311528565.2

(51) Int. Cl. A61H 7/00; A61H 23/02

(52) U.S. Cl. CPC A61H 7/005; A61H 23/02; A61H 2201/0107; A61H 2201/0188; A61H 2201/0207; A61H 2201/1215; A61H 2201/149; A61H 2201/1669; A61H 2201/5066

(57) ABSTRACT

The present invention discloses a massager capable of adjusting a range of motion of a massaging member, which comprises the massaging member, a reciprocating translation driving device and a range of motion adjustment module. The range of motion adjustment module adjusts a range of motion of a reciprocating translation member of the reciprocating translation driving device so as to massage a position on a massage area where massage is required, improving the massage experience effect.

### TECHNICAL FIELD

The present invention pertains to the technical field of massage instruments, and specifically pertains to a massager capable of adjusting a range of motion of a massaging member.

### BACKGROUND

Massagers have been widely used and popularized due to their remarkable efficacy, such as massagers in which massage sleeves are driven by reciprocating translation driving devices to perform reciprocating linear motion in the prior art. However, when such a massager works, the massage sleeve is subject to a full-stroke linear reciprocating motion, so that the position on the massage area where the massage is not required is also massaged, thereby affecting the massage experience effect. Therefore, there is a need for improvement.

### SUMMARY

It is an object of the present invention to design a massager capable of adjusting a range of motion of a massaging member in order to solve the above-mentioned shortcomings of the art.

The present invention provides a massager capable of adjusting a range of motion of a massaging member, comprises:
the massaging member;
a reciprocating translation driving device, wherein the reciprocating translation driving device comprises a reciprocating translation member, a sensing member is mounted on the reciprocating translation member, the massaging member is mounted on the reciprocating translation member, and when the reciprocating translation driving device (2) drives the reciprocating translation member (23) to perform a linear reciprocating translation motion, the massaging member and the sensing member are driven to perform linear reciprocating translation motion synchronously; and
a range of motion adjustment module, wherein the range of motion adjustment module comprises a main control module and a sensing module, a position of the sensing member corresponds to a position of the sensing module, the sensing module comprises at least three limit sensors arranged at intervals, the at least three limit sensors are respectively connected with the main control module to control two limit sensors to work, and the at least three limit sensors are arranged along a motion track direction of the sensing member so that the sensing member is capable of reaching the side of a sensing portion of any of the limit sensors, and a distance between the two limit sensors serves as the range of motion of the massaging member.

The massager capable of adjusting the range of motion of the massaging member according to the present invention has the following beneficial effects:
1. The massager operates in a mode in which any two limit sensors are active and the remaining limit sensor is inactive to define the stroke and range of motion of the massaging member so as to massage the position on the massage area where massage is required, thereby further enhancing the massage experience effect.
2. The turbine is driven by two motors and two worms to rotate, so that the rotating member rotates stably and reliably, which improving stability of the massager.
3. The vibration motor and the electric heating sheet are mounted on the flexible massage sleeve to provide an additional function of vibrating or heating massage during the massage to enhance the comfort of the massage.
4. The range of motion of the massaging member can be observed through the scale, and light can be transmitted out of the light transmission scale through the operation of the light board, so that the massager looks aesthetically pleasing in use.
5. The two hand-held bodies can be turned to an unfolded state, so that the massager can be stably held when the two hand-held bodies are held by two hands, thereby facilitating hand-held massage.

### BRIEF DESCRIPTION OF THE DRAWINGS

FIG. 1 is a schematic view of an overall structure;

FIG. 2 is a sectional view of the overall structure (I);

FIG. 3 is a schematic view (I) showing an internal structure with an outer casing hidden;

FIG. 4 is a partial structural schematic view of a reciprocating translation driving device;

FIG. 5 is a schematic view showing a structure in which a rotation driving mechanism is combined with a rotating member;

FIG. 6 is a schematic view showing an internal structure of the rotation driving mechanism with a lower half casing and a lower casing hidden;

FIG. 7 is a schematic view (II) showing the internal structure with the outer casing hidden;

FIG. 8 is an exploded view (I);

FIG. 9 is an exploded view (II);

FIG. 10 is an exploded view (III); and

FIG. 11 is a sectional view of the overall structure (II).

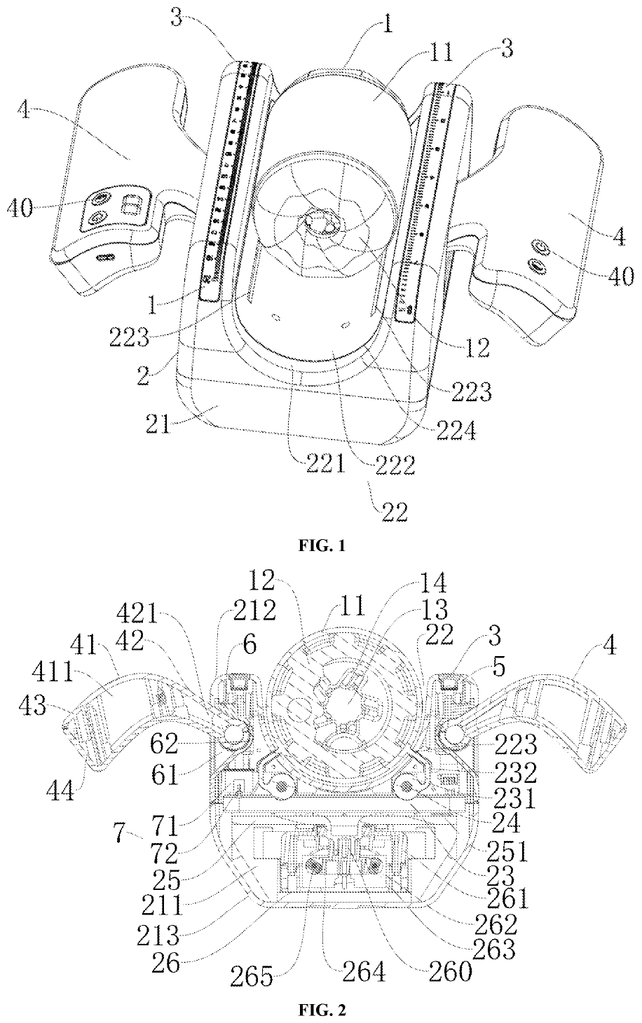

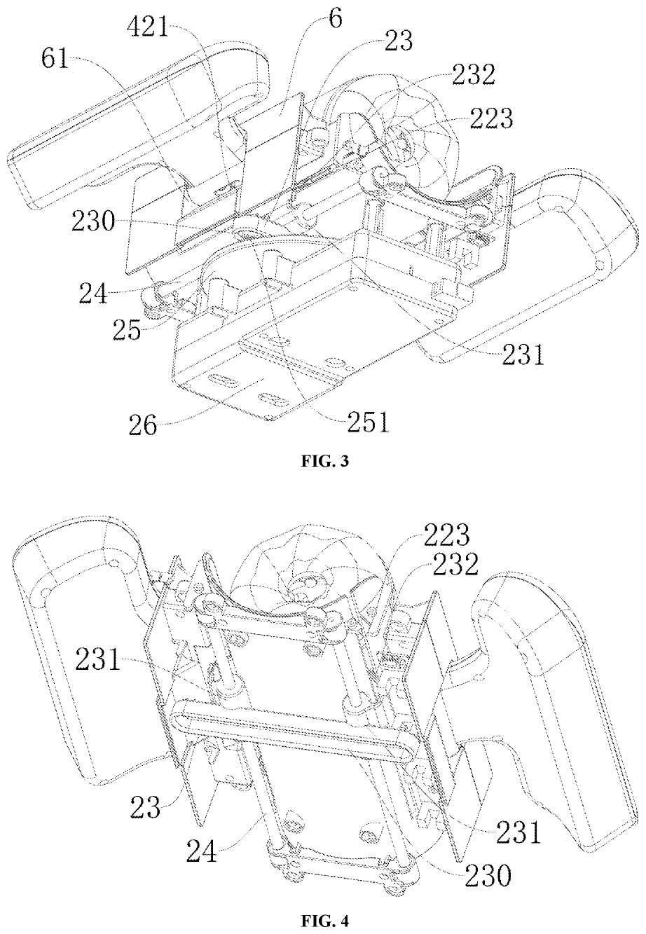

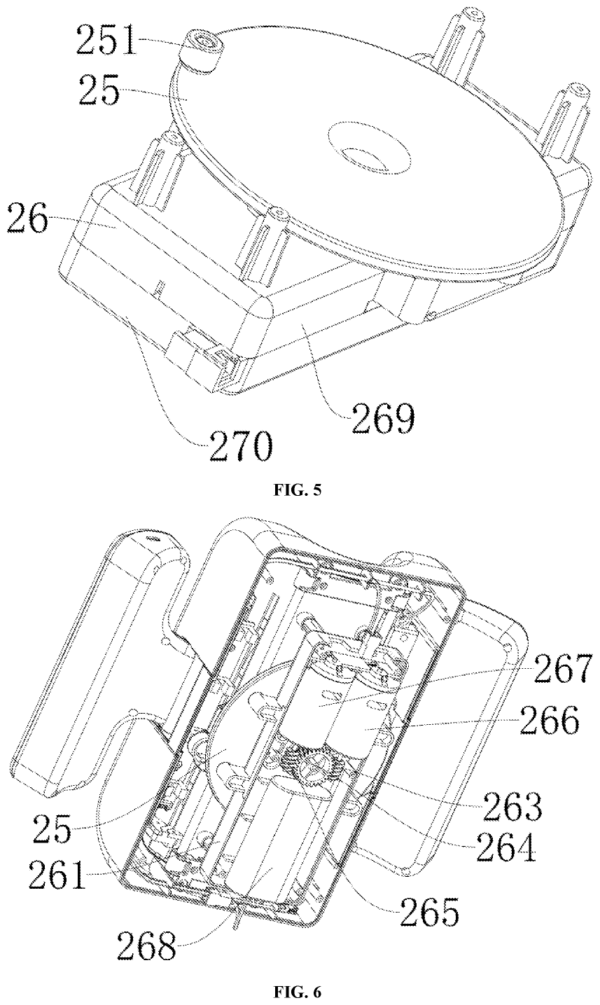

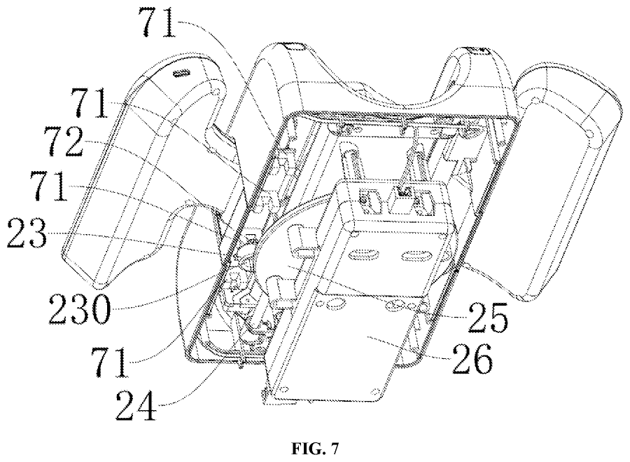

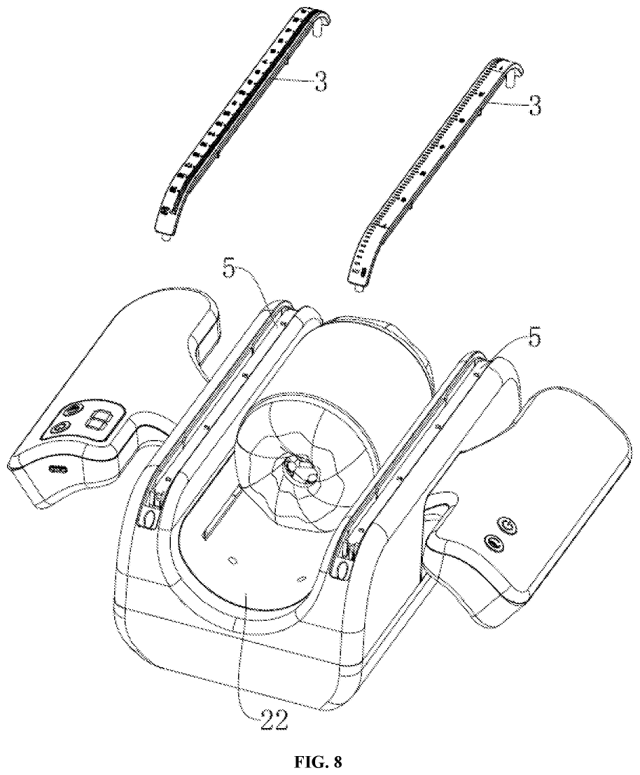

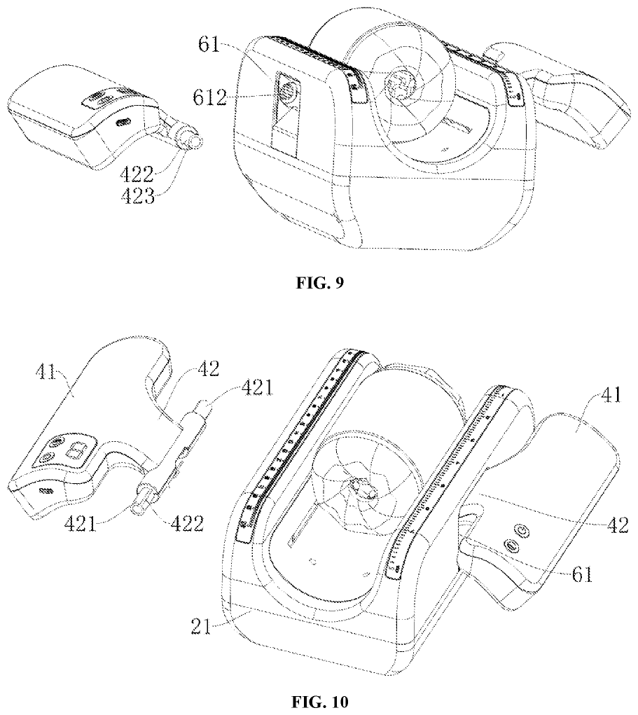

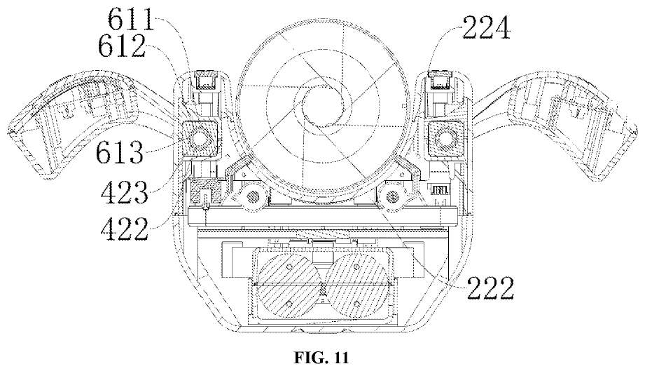

Reference numerals: 1. massaging member; 11. cylinder; 12. flexible massage sleeve; 13. massage channel; 14. flexible massage protrusion; 2. reciprocating translation driving device; 21. outer casing; 211. inner cavity; 212. upper half casing; 213. lower half casing; 22. guide portion; 221, elongated recess; 222. elongated arc-shaped plate; 223. elongated hole; 224. mounting hole; 23. reciprocating translation member; 230. elongated groove; 231. guide sleeve; 232. guide body; 24. guide rod; 25. rotating member; 251. eccentric shaft; 26. rotation driving mechanism; 260, rotating shaft; 261. inner casing; 262. mounting cavity; 263. first worm; 264. turbine; 265. second worm; 266. first motor; 267. second motor; 268. battery; 269. upper elongated casing; 270. lower elongated casing; 3. light transmission scale; 5. light board; 4. hand-held body; 40. control key; 41. hand-held portion; 411. cavity body; 42. hinging portion; 421. passing channel; 422. hinged shaft; 423. locking protrusion; 43. upper casing; 44. lower casing; 6. hinging member; 61. hinging groove; 611. hinging hole; 612. locking teeth; 613. locking groove; 62. passing mouth; 7. range of motion adjustment module; 71. limit sensor; and 72. sensing member.

### DESCRIPTION OF THE EMBODIMENTS

The present invention will be further described with reference to the accompanying drawings and embodiments.

As shown in the figures, a massager capable of adjusting a range of motion of a massaging member 1, comprises the massaging member 1, a reciprocating translation driving device 2 and a range of motion adjustment module 7. The range of motion adjustment module 7 is used to adjust and control a range of motion of a reciprocating translation member 23 of the reciprocating translation driving device 2 so as to control the range of motion of the massaging member 1 and massage a position on a massage area where massage is required.

The reciprocating translation driving device 2 comprises the reciprocating translation member 23, an outer casing 21, a rotating member 25, a rotation driving mechanism 26 and a guide rod 24. a sensing member 72 is mounted on the reciprocating translation member 23, an inner cavity 211 and a guide portion 22 are provided on the outer casing 21, the guide portion 22 and the guide rod 24 are both arranged along a length direction of the outer casing 21, and the rotating member 25, the rotation driving mechanism 26 and the guide rod 24 are all located in the inner cavity 211; the guide rod 24 and the rotation driving mechanism 26 are both fixed on an inner wall of the inner cavity 211, the guide portion 22 and the guide rod 24 are arranged parallel to each other, the reciprocating translation member 23 is located between the guide rod 24 and the rotating member 25, a guide sleeve 231 and an elongated groove 230 provided along a width direction of the outer casing 21 are provided on the reciprocating translation member 23, an eccentric shaft 251 is provided on an eccentric position of the rotating member 25, the eccentric shaft 251 is inserted into the elongated groove 230, and a bearing can be sleeved on the eccentric shaft 251; and an outer wall of the bearing is in contact with an inner wall of the elongated groove 230 so as to play the role of rolling guide; the guide sleeve 231 is sheathed on the guide rod 24, a rotating shaft 260 of the rotation driving mechanism 26 is connected to a central position of the rotating member 25, the rotating member 25 is of a disc-shaped structure, the massaging member 1 is movably fitted on the guide portion 22, an elongated hole 223 is provided on the guide portion 22, the elongated hole 223 is arranged along the length direction of the outer casing 21, and a guide body 232 on the guide sleeve 231 passes through the elongated hole 223 and is then connected to the massaging member 1 so as to mount the massaging member 1 on the reciprocating translation member 23; a linear motion track of the sensing member 72 and the massaging member 1 is arranged along the length direction of the outer casing 21; the rotation driving mechanism 26 comprises an inner casing 261, a battery 268, a first motor 266, a turbine 264 and a first worm 263; a mounting cavity 262 is provided on the inner casing 261; the battery 268, the first motor 266, the turbine 264 and the first worm 263 are all located in the mounting cavity 262; the rotating shaft 260 is rotatably mounted on a side of the casing facing the rotating member 25, the rotating shaft 260 is mounted at a central position of the turbine 264, the first motor 266 and the battery 268 are both fixed to an inner wall of the mounting cavity 262, the first worm 263 is mounted on a rotating shaft of the first motor 266, and the first worm 263 is intermeshed with the turbine 264 for transmission; and in order to improve stability of linear reciprocating translation motion of the reciprocating translation member 23, two guide rods 24 can be provided, and two guide sleeves 231, two guide bodies 232 and two elongated hole 223 can be provided, and the two guide sleeves 231, the reciprocating translation member 23 and the guide bodies 232 on each of the guide sleeves 231 are integrated with each other into an integral reciprocating translation member 23.

Further, a length of the guide portion 22 and a length of the elongated hole 223 are greater than a length of the linear motion track of the sensing member 72, and the guide portion 22 is an elongated guide channel formed by recessing, which is provided to enable the massaging member 1 to move within the range of the outer casing 21.

The first worm 263 rotates when the first motor 266 operates, and intermeshing transmission between the first worm 263 and the turbine 264 drives the rotating shaft 260 to rotate, and then the eccentric shaft 251 is driven to circularly move around an axis of the rotating shaft 260, in this case, the eccentric shaft 251 drives the reciprocating translation member 23 to perform a linear reciprocating motion under the guide of the elongated groove 230 and the mating guide of the guide rod 24 and the guide sleeve 231, the massaging member 1 and the sensing member 72 are synchronously driven to perform the linear reciprocating translation motion, and the sensing member 72 is a baffle structure.

Preferably, the rotation driving mechanism 26 further comprises a second motor 267 and a second worm 265, the second motor 267 is fixed to the inner wall of the mounting cavity 262, the second worm 265 is mounted on a rotating shaft of the second motor 267, and the second worm 265 is intermeshed with the turbine 264 for transmission, wherein the first motor 266 and the second motor 267 operate synchronously to drive the first worm 263 and the second worm 265 to operate, thereby driving the turbine 264 to rotate in such a manner that the rotating member 25 rotates more stably and reliably, and the first motor 266 and the second motor 267 are both powered by the battery 268.

The range of motion adjustment module 7 comprises a main control module and a sensing module, a position of the sensing member 72 corresponds to a position of the sensing module, the sensing module comprises at least three limit sensors 71 arranged at intervals, the at least three limit sensors 71 are respectively connected with the main control module to control only two limit sensors 71 to work, and the at least three limit sensors 71 are arranged along the linear motion track direction of the sensing member 72 so that the sensing member 72 can reach the side of a sensing portion of any of the limit sensors 71; when the three limit sensors 71 are used, the main control module can control the limit sensors 71 at a head end and a tail end to operate, and a distance between the two limit sensors 71 serves as the range of motion of the massaging member 1 so as to form a first adjustment mode, or the main control module controls two adjacent limit sensors 71 to operate, and a distance between the two limit sensors 71 serves as the range of motion of the massaging member 1 so as to form a second adjustment mode; in the first adjustment mode, the rotating member 25 rotates continuously by 360°; and in the second adjustment mode, the rotating member 25 rotates back and forth by 90° or less, and an angle of rotation depends on the distance between the two adjacent limit sensors 71, and generally can be set according to implementation designs.

Preferably, the number of limit sensors 71 can also be increased to four, because four limit sensors 71 allows more range of motion adjustment modes for the massaging member 1; when four limit sensors 71 are used, the main control module controls the limit sensors 71 at the head end and the tail end to operate, and the distance between the two limit sensors 71 serves as the range of motion of the massaging member 1 to form a first adjustment mode, or the main control module controls two adjacent limit sensors 71 to operate, and a distance between the two limit sensors 71 serves as the range of motion of the massaging member 1 to form a second adjustment mode; or the main control module controls the limit sensor 71 at the head end and the tail end and the limit sensor 71 near the tail end at a middle position to operate, and the distance between the two limit sensors 71 serves as the range of motion of the massaging member 1 to form a third adjustment mode; or the main control module controls the limit sensor 71 at the tail end and the limit sensor 71 near the head end at the middle position to operate, and a distance between the two limit sensors 71 serves as the range of motion of the massaging member 1 so as to form a fourth adjustment mode; in the first adjustment mode, the rotating member 25 rotates continuously by 360°; in the second adjustment mode, the rotating member 25 rotates back and forth by less than 90°; in the third adjustment mode or the fourth adjustment mode, the rotating member 25 rotates back and forth by 90°; and in the second adjustment mode, the third adjustment mode and the fourth adjustment mode, the angle of rotation of the rotating member 25 depends on the distance between the two limit sensors 71, and generally can be set according to the implementation designs.

Based on the above-mentioned features, the main control module is a CPU chip or a MCU chip, all the limit sensors 71 are photoelectric sensors or contact sensors, and the main control module has a built-in program for controlling the light board 5, the two motors and all the limit sensors 71 to be turned on or off respectively; therefore, in any of the adjustment modes, the sensing member 72 is driven between the two limit sensors 71, and then when the sensing member 72 is located beside the sensing portion of one limit sensor 71, the limit sensor 71 transmits a sensing signal to the main control module; and the main control module controls rotation directions of the rotating shafts of the two motors to reverse, causing the sensing member 72 and the massaging member 1 to move towards the other limit sensor 71. However, when the sensing member 72 is located beside the sensing portion of one limit sensor 71, the limit sensor 71 transmits the sensing signal to the main control module, and the main control module controls the rotation direction of the rotating shafts of the two motors to reverse again, causing the sensing member 72 and the massaging member 1 to move in an opposite direction again, thereby realizing the linear reciprocating translation motion of the massaging member 1 and the sensing member 72 between the two limit sensors 71 in an adjustment mode, and further controlling the massaging member 1 to perform the linear reciprocating translation motion in a certain range of motion on the guide portion 22 according to the mode adjustment.

In the present embodiment, the massaging member 1 comprises a cylinder 11 and a flexible massage sleeve 12, a mounting channel is provided on the cylinder 11, the flexible massage sleeve 12 is mounted in the mounting channel, a massage channel 13 is provided on the flexible massage sleeve 12, a plurality of flexible massage protrusions 14 are formed on an inner wall of the massage channel 13, the flexible massage sleeve 12 and the flexible massage protrusions 14 are combined into an integral silicone massage cylinder, a guide body 232 on the guide sleeve 231 is connected with the cylinder 11, a groove bottom of an elongated groove is an arc surface, and the arc surface is matched with the cylinder 11.

Preferably, a vibration motor and/or an electric heating sheet are/is mounted on the flexible massage sleeve 12, the vibration motor and/or the electric heating sheet are/is connected with the main control module, the vibration motor provides a vibration function for the silicone massage cylinder so as to realize vibration massage, and the electric heating sheet provides a heating function for the silicone massage cylinder so as to realize heating massage, wherein the vibration motor and the electric heating sheet are both powered by the battery 268.

In the present embodiment, a light transmission scale 3 is provided beside the guide portion 22, the light transmission scale 3 is arranged along the length direction of the outer casing 21, the guide portion 22 and the light transmission scale 3 are arranged parallel to each other, a light board 5 is mounted on a bottom surface of the light transmission scale, the light board 5 is connected with a main control module, the light board 5 is of an LED light board, and the battery 268 powers the LED light board; and when lit on, the light transmission scale 3 transmits light, so that the massager looks aesthetically pleasing in use; and the light transmission scale 3 and the light board 5 are combined with each other to form a light module. Two light modules may be provided, and the two light modules are respectively fixed to left and right sides of the guide portion 22.

In the present embodiment, the massager further comprises two hand-held bodies 4, the hand-held bodies 4 are respectively located on a left side and a right side of the outer casing 21 in the length direction, the left side and the right side of the outer casing 21 in the length direction are respectively recessed to form hinging grooves 61, each of the hand-held bodies 4 comprises a hand-held portion 41 and a hinging portion 42 corresponding to a position of the hinging groove 61, the hinging portion 42 is convexly arranged on a side portion of the hand-held portion 41 facing the outer casing 21, and a hinging end of the hinging portion 42 is inserted into the hinging groove 61; a front end face and a rear end face of the hinging groove 61 are respectively recessed to form hinging holes 611, a convex hinged shaft 422 is respectively formed on a front end face and a rear end face of a hinged end of the hinging portion 42, the hinged shaft 422 is inserted into the hinging hole 611, an inner wall of the hinging hole 611 is provided with a plurality of locking teeth 612 arranged in an annular array, and a locking groove 613 is formed between every two adjacent locking teeth 612; and an outer side wall of the hinged shaft 422 is provided with a locking protrusion 423, and the locking protrusion 423 is located in the locking groove 613 to position a turning angle of the hand-held bodies 4 after turning. The hinging hole 611 fits with the hinged shaft 422, allowing the two hand-held bodies 4 to turn on the outer casing 21; and the locking protrusion 423 fits with the locking groove 613, enabling the hand-held bodies 4 to be positioned at any angle, so that the two hand-held bodies 4 can be unfolded when hand-holding is desired, the two hand-held bodies can be folded when storage is desired, and an outer wall of the locking protrusion 423 and an inner wall of the locking groove 613 are both circular arc surfaces.

Further, a cavity body 411 is provided on the hand-held portion 41, a passing channel 421 is provided on the hinging portion 42, a passing mouth 62 communicating with the inner cavity 211 is formed on an inner wall of the hinging groove 61, the passing mouth 62 corresponds to and communicates with a port of the passing channel 421, a circuit board is mounted in the cavity body 411 of one of the hand-held bodies 4, the main control module is arranged on the circuit board, and conductive wires on the first motor 266, conductive wires on the second motor 267, conductive wires on the battery 268 and conductive wires on the sensing module sequentially pass through the passing mouth 62 and the passing channel 421 into the cavity body 421 to be connected with the main control module; and a control key 40 is provided in the cavity body 411 of the other hand-held body 4 and on the circuit board, the control key runs through the wall thickness of the cavity body 411 to be exposed to the outside, and the control key 40 on the other hand-held body 4 is connected with the main control module.

Preferably, the outer casing 21 is composed of an elongated arc-shaped plate 222, an upper half casing 212 and a lower half casing 213; the upper half casing 212 and the lower half casing 213 are assembled and then fixed by bolts, a left side and a right side of the upper half casing 212 form hinging openings respectively, a hinging member 6 is provided at each hinging opening, the hinging member 6 is fixed on an inner wall of the upper half casing 212 by bolts, and the hinging groove 61 is formed on the hinging member 6; the four limit sensors 71 are also fixed on a bottom surface of the hinging member by the circuit board, and the circuit board is fixed to the bottom surface of the hinging member 6 by bolts; a top surface of the upper half casing 212 is provided with an elongated recess 221, a bottom surface of the elongated recess 221 is provided with a mounting hole 224 matching the shape of the elongated arc-shaped plate 222, the elongated arc-shaped plate 222 is fitted and mounted in the mounting hole 224, the elongated arc-shaped plate 222 is fixed to the inner casing 261 by bolts, the elongated hole 223 is arranged on the elongated arc-shaped plate 222, and the elongated arc-shaped plate 222 and the elongated recess 221 are combined to form an elongated groove; and the inner casing 261 is composed of an upper elongated casing 269 and a lower elongated casing 270, and the upper elongated casing 269 and the lower elongated casing 270 are symmetrically assembled with each other and fixed by bolts.

The two hand-held bodies 4 are also composed of an upper casing 43 and a lower casing 44, and the upper casing 43 and the lower casing 44 are assembled with each other and fixed by bolts.

What is claimed is:

1. A massager capable of adjusting a range of motion of a massaging member, comprising:
the massaging member;
a reciprocating translation driving device, wherein the reciprocating translation driving device comprises a reciprocating translation member, a sensing member is mounted on the reciprocating translation member, the massaging member is mounted on the reciprocating translation member, and when the reciprocating translation driving device drives the reciprocating translation member to perform a linear reciprocating translation motion, the massaging member and the sensing member are driven to perform linear reciprocating translation motion synchronously; and
a range of motion adjustment module, wherein the range of motion adjustment module comprises a main control module and a sensing module, a position of the sensing member corresponds to a position of the sensing module, the sensing module comprises at least three limit sensors arranged at intervals, the at least three limit sensors are respectively connected with the main control module to control two limit sensors to operate, and the at least three limit sensors are arranged along a motion track direction of the sensing member so that the sensing member is capable of reaching the side of a sensing portion of any of the limit sensors, and a distance between the two limit sensors serves as the range of motion of the massaging member.

2. The massager according to claim 1, wherein the reciprocating translation driving device further comprises an outer casing, a rotating member, a rotation driving mechanism and a guide rod; an inner cavity and a guide portion are provided on the outer casing, the guide portion and the guide rod are both arranged along a length direction of the outer casing, the rotating member, the rotation driving mechanism and the guide rod are all located in the inner cavity, the guide rod and the rotation driving mechanism are both fixed on an inner wall of the inner cavity, and the guide portion and the guide rod are arranged parallel to each other; the reciprocating translation member is located between the guide rod and the rotating member, a guide sleeve and an elongated groove provided along a width direction of the outer casing are provided on the reciprocating translation member, an eccentric shaft is provided on an eccentric position of the rotating member, the eccentric shaft is inserted into the elongated groove, the guide sleeve is sheathed on the guide rod, a rotating shaft of the rotation driving mechanism is connected to a central position of the rotating member, the massaging member is movably fitted on the guide portion, and an elongated hole is provided on the guide portion; and the elongated hole is arranged along the length direction of the outer casing, a guide body on the guide sleeve passes through the elongated hole and is then connected to the massaging member, and a linear motion track of the sensing member and the massaging member is arranged along the length direction of the outer casing.

3. The massager according to claim 2, wherein the rotation driving mechanism comprises an inner casing, a battery, a first motor, a turbine and a first worm; a mounting cavity is provided on the inner casing, the battery, the first motor, the turbine and the first worm are all located in the mounting cavity, the rotating shaft is rotatably mounted on a side of the casing facing the rotating member, the rotating shaft is mounted at a central position of the turbine, the first motor and the battery are both fixed to an inner wall of the mounting cavity, the first worm is mounted on the rotating shaft of the first motor, and the first worm is intermeshed with the turbine for transmission.

4. The massager according to claim 3, wherein the rotation driving mechanism further comprises a second motor and a second worm, the second motor is fixed to the inner wall of the mounting cavity, the second worm is mounted on a rotating shaft of the second motor, and the second worm is intermeshed with the worm for transmission.

5. The massager according to claim 2, wherein a length of the guide portion and a length of the elongated hole are greater than a length of the linear motion track of the sensing member, and the guide portion is an elongated guide channel formed by recessing.

6. The massager according to claim 5, wherein the massaging member comprises a cylinder and a flexible massage sleeve, a mounting channel is provided on the cylinder, the flexible massage sleeve is mounted in the mounting channel, a massage channel is provided on the flexible massage sleeve, a plurality of flexible massage protrusions are formed on an inner wall of the massage channel, and the guide body on the guide sleeve is connected with the cylinder.

7. The massager according to claim 6, wherein a vibration motor or an electric heating sheet is mounted on the flexible massage sleeve, and the vibration motor or the electric heating sheet is connected with the main control module.

8. The massager according to claim 5, wherein a light transmission scale is provided beside the guide portion, the light transmission scale is arranged along the length direction of the outer casing, the guide portion and the light transmission scale are arranged parallel to each other, and a light board is mounted on a bottom surface of the light transmission scale, and the light board is connected with the main control module.

9. The massager according to claim 6, wherein the massager further comprises two hand-held bodies, the hand-held bodies are respectively located on a left side and a right side of the outer casing in the length direction, the left side and the right side of the outer casing in the length direction are respectively recessed to form hinging grooves, each of the hand-held bodies comprises a hand-held portion and a hinging portion corresponding to a position of the hinging groove, the hinging portion is convexly arranged on a side portion of the hand-held portion facing the outer casing, and a hinging end of the hinging portion is inserted into the hinging groove; a front end face and a rear end face of the hinging groove are respectively recessed to form hinging holes, a convex hinged shaft is respectively formed on a front end face and a rear end face of a hinged end of the hinging portion, the hinged shaft is inserted into the hinging hole, an inner wall of the hinging hole is provided with a plurality of locking teeth arranged in an annular array, and a locking groove is formed between every two adjacent locking teeth; and an outer side wall of the hinged shaft is provided with a locking protrusion, and the locking protrusion is located in the locking groove to position a turning angle of the hand-held bodies after turning.

10. The massager according to claim 1, wherein the number of limit sensors is four.

11. The massager according to claim 9, wherein a cavity body is provided on the hand-held portion, a passing channel is provided on the hinging portion, a passing mouth communicating with the inner cavity is formed on an inner wall of the hinging groove, the passing mouth corresponds to and communicates with a port of the passing channel, a circuit board is mounted in the cavity body of one of the hand-held bodies, the main control module is arranged on the circuit board, and conductive wires on the first motor, conductive wires on the second motor, conductive wires on the battery and conductive wires on the sensing module sequentially pass through the passing mouth and the passing channel into the cavity body to be connected with the main control module.
